(19) United States
(12) Patent Application Publication
(10) Pub. No.: US 2002/0106708 A1
(43) Pub. Date: Aug. 8, 2002

(54) Assays reagents and kits for detecting or determining the concentration of analytes

(75) Inventors: Linda Thomas, Ballwin, MO (US); Venita Eggerding, St. Louis, MO (US); Mark X. Triscott, Raleigh, NC (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Sigma-Aldrich Co.

(21) Appl. No.: 10/012,280

(22) Filed: Nov. 13, 2001

Related U.S. Application Data
(60) Provisional application No. 60/248,116, filed on Nov. 13, 2000.

(51) Int. Cl. G01N 33/53; G01N 33/542; G01N 33/537; G01N 33/543

(52) U.S. Cl. 435/7.93

(57) ABSTRACT

Non-competitive immunoassays and kits for detecting or determining the concentration of an analyte in a biological sample are provided along with reagents for use in such assays and kits. Immunoassays are provided which employ a reagent containing an autologous analyte which raises the signal above the background noise caused by non-specific binding thereby lowering the percent coefficient of variation of a non-competitive assay. By employing an autologous analyte in a reagent of the immunoassay, low dosages of an analyte can be detected without adversely affecting the long linear range of the assay.

This application claims priority to co-pending U.S. provisional patent application Serial No. 60/248,116, filed Nov. 13, 2000, incorporated herein by reference.

### BACKGROUND OF THE INVENTION

The present invention generally relates to diagnostic non-competitive immunoassays, and specifically, to reagents suitable for use in connection therewith. The invention particularly relates to non-competitive immunoassays for detecting the presence and/or the amount of an analyte in a sample.

A wide variety of immunological detection methods based on the agglutination principle exist for detecting the presence or the concentration of a target analyte in a biological specimen. Typically, a biological sample suspected of containing an analyte is combined with a reagent containing a receptor or binding partner directed against the analyte under conditions in which an agglutination of the reaction partners takes place. The agglutination caused by the immunological reaction leads to particle aggregates whose light scattering properties differ from those of the starting substances thus enabling the analyte to be detected and the concentration thereof to be determined.

Microparticles are commonly utilized in agglutination immunoassays. In these immunoassays, microparticles are coated with monoclonal and/or polyclonal antibodies which are selected to react with at least two non-overlapping epitopes of the analyte of interest. These antibody coated particles are introduced to a sample which may be diluted in a reaction buffer. Once in the presence of the analyte of interest, the antibody-coated particles will agglutinate thereby initiating a light scattering event which is detected by appropriate instrumentation. One example of an instrument is a spectrophotometer which converts a light signal (absorbance or transmission) into an electrical signal (often measured in mE).

Agglutination immunoassays may be used to determine whether a threshold or minimal level (“the cutoff point”) of a particular analyte is present in a biological sample. If the analyte is below the cutoff point, a particular path of patient management is taken; however, if the analyte is above the cut off point, another path of treatment is taken. For example, TRIAGE® assays are used to determine the concentration levels of various cardiac markers such as myoglobin, creatine kinase MB (CK-MB), and troponin I in order to assess for cardiac damage in a patient. If certain markers are above the cutoff point, the patient is treated for suspected myocardial infarction. If, however, the levels of certain cardiac markers are below the cutoff point, other causes for the pain will be investigated and the patient will not be admitted for expensive cardiac related treatment.

Additionally, immunological agglutination assays are used clinically for screening patients' plasma for coagulation deficiencies. For instance, fibrin d-dimer may be measured in an emergency room as part of an algorithm in the diagnosis for deep venous thrombosis (DVT) or pulmonary embolus (PE). If the concentration of d-dimer is above a certain threshold, the possibility exists that the patient may be suffering from one of these coagulation disorders. However, if the levels of d-dimer are below the threshold cutoff point, there is a good chance that the patient is not suffering from these conditions. In some cases, the concentration of the analyte in the patient sample may only be slightly lower than the threshold value for the cutoff point. Thus, when the threshold cutoff point for the target analyte in the sample is also low, it is often difficult to distinguish between various low concentrations of analyte in a sample in order to obtain an accurate diagnosis. It is in these cases that the precision of the determination of analyte concentration is critical in order to correctly determine patient management options.

Further, the sample may include low levels of a protein or other moiety unrelated to the target analyte which participates in non-specific binding reactions with the various components of the assay (e.g., the antibody). Such non-specific binding reactions between a non-target moiety and the antibody is a factor which results in false positives (FP) and false negatives (FN) for the results of the assay. A high level of false positives and false negatives impacts the sensitivity and precision of an assay. The sensitivity of an assay is determined by the formula: TP/(TP+FN); where TP represents the number of true positive results and FN is the number of false negative results. The specificity of an assay is determined by the formula TN/(TN+FN); where TN is the number of true negatives and FN is the number of false negatives. Thus, reducing the number of false negatives and false positives will positively affect the sensitivity and specificity of an immunoassay.

Accordingly, desirable parameters of an immunoassay include the reportable range of the assay and precision. Using currently available immunoassays, in order to get a long linear range for the assay, the clinician has to accept a low signal for low doses of the analyte.

Specifically, a low signal is obtained by the assay instrumentation when the signal is less than 5% of the total instrument range. Thus, utilizing a spectrophotometer to determine the concentration of d-dimer (total signal range is 2-3 “E” units) may result in a measurement range for the bottom (i.e., measurement of low dosages) of a d-dimer dosage curve as low as 6 or 7 milli E which is approximately less than 1% of the total signal range for the instrument.

Furthermore, these low signals give unacceptably high coefficients of variation which result from a number of fixed sources of variability associated with running the assay. For example, if an automatic pipetting instrument is used, there may a 5% variation in all of the pipetting steps. Cuvettes placed in the instrumentation light path may have manufacturing variabilities which contribute to a small percentage of variation in light transmission. Further, the spectrophotmeter may have a variability in the signal output from the photomultiplier thus resulting in another fixed amount of variability.

The percentage variation of the assay is measured by the coefficient of variation (CV) which is determined by the formula: % CV=δ/\overline{x}×100; where δ represents the standard of deviation and \overline{x} represents the mean. Higher values for the % CV indicate a lower 20 confidence level for the results of the assay. The confidence level can be determined by statistical analysis of biological samples containing appropriate ranges of analyte concentrations. In most clinical settings, a coefficient of variation above 20% is often considered to be unacceptable. A low coefficient of variation is seen as an desirable parameter in an immunoassay where the designated value for the cutoff point will have an effect on patient management.

Accordingly, it would be desirable to provide reagents for use in immunoassays which result in an increase in the low end signal without adversely affecting the long linear range of the assay. Assays which could be employed to detect low dosages of an analyte in a sample with improved coefficients of variation would be a great improvement over assays and kits currently available in the art.

### SUMMARY OF THE INVENTION

Accordingly, it is therefore an object of the present invention to provide immunoassays and kits which can be used to detect or determine the concentration of an analyte in a sample with a low coefficient of variation thereby increasing the assay precision and confidence level. It is another object to provide assays and kits having improved precision by which it is possible to measure low dosages of an analyte in a biological sample. The kits and assays of the present invention employ a reagent which reduces the rate of false positives (FP) and false negatives (FN), thereby increasing the sensitivity and precision of the assay.

Briefly, therefore, the present invention is directed to processes for detecting or determining the concentration of a target analyte in a sample. Such processes generally include combining the sample with a reagent to form an assay mixture. The reagent used in the immunoassay contains an amount of target analyte from a source other than the sample. Preferably, the amount of target analyte in the reagent is in an amount which lowers a percent coefficient of variation for the target analyte in a non-competitive assay by at least about 10% relative to the results of an non-competitive immunoassay run without the use of a reagent containing the target analyte. In one embodiment, autologous analyte is added to the reagent in an amount to increase the signal of the immunoassay for low dosages of the target analyte in the sample by at least about 50% of the expected value. Low dosages of target analyte are preferably about 70 to about 150 ng/ml of d-dimer or about 0.2 to about 0.8 ng/ml of troponin I. The expected value is the value from a dosage-response curve w/o autologous analyte wherein the value is greater than zero.

The assay mixture is then incubated with an antibody mixture containing antibodies which bind to the target analyte. Preferably, the antibodies in the antibody mixture are monoclonal antibodies. In a preferred embodiment, the antibody mixture contains antibodies which bind to at least two non-overlapping epitopes on the target analyte. In another preferred embodiment, the antibody mixture contains antibodies immobilized to latex particles such as latex beads. The amount of analyte bound to the antibodies can then be detected by conventional techniques and related to the presence or concentration of analyte in the sample.

It is a further object of the present invention to provide kits for conducting a non-competitive immunoassay for detecting or determining the concentration of a target analyte in a sample. Kits of the present invention contain a reagent containing the target analyte derived from a source other than the sample and an antibody mixture comprising antibodies which bind to the target analyte. Preferably, the amount of target analyte in the reagent is in an amount which lowers the percent coefficient of variation for the target analyte in a non-competitive assay by at least about 10% relative to the results of an non-competitive immunoassay run without the use of a reagent containing the target analyte. Preferably, the antibodies contained in the antibody mixture are monoclonal antibodies. In a preferred embodiment, the antibody mixture contains antibodies which bind to at least two non-overlapping epitopes on the target analyte. In another preferred embodiment of the invention, the antibody mixture contains antibodies immobilized to latex particles such as latex beads.

It will be appreciated that by virtue of the present invention, the target analyte being assayed by the processes and kits as disclosed herein may be a number of various analytes. In a preferred embodiment, the sample is being assayed for the presence and/or concentration of d-dimer or troponin I.

The immunoassays and kits of the present invention offer significant advantages over prior art assays by employing a reagent which increases the sensitivity and precision of the assay. Accordingly, it is a related object of the present invention to provide a reagent for use in a non-competitive immunoassay for an analyte in a sample. The reagent of the present invention contains the analyte being assayed for which is derived from a source other than the sample. Further, the analyte is dissolved in a solution other than the sample in an amount which lowers a percent coefficient of variation for the analyte in the non-competitive assay by at least about 10% relative to the non-competitive immunoassay run without the use of a reagent containing an amount of analyte derived from a source other than the sample.

Other objects and features will be in part apparent and in part pointed out hereinafter.

### BRIEF DESCRIPTION OF THE DRAWINGS

FIG. 1 is a graph illustrating calibration curves of an assay using a reaction buffer containing various concentrations of d-dimer.

FIG. 2 is a graph illustrating the comparison of calibration curves of an assay which employs a reaction buffer with the addition of 200 ng/ml of d-dimer with an assay which employs a reaction buffer without the addition of d-dimer.

### DEFINITIONS AND ABBREVIATIONS

To facilitate understanding of the invention, a number of terms are defined below:

An “antibody” is a specific binding protein with multiple defined tertiary and quaternary structure. Antibodies have isotypic variations which are structural variations exhibited in the so-called constant region of their structure which allows them to be segregated into classes which include the IgG, IgA, IgM, IgD and IgE classes (so called isotypes). Idiotypic variations exhibited by antibodies within a given class in the hypervariable regions of their structure determine the shape and structure of the binding site, and thus determine the specificity of the antibody. A “monoclonal antibody” is an antibody molecule arising from a single clone of antibody-producing cells having the same idiotypic and isotypic structure.

An “analyte” is any composition found in a biological sample for which it would be useful to detect its presence and/or quantity. The analytes used in the reagents of the present invention can be any medically or biologically significant composition for which the presence or quantity thereof in a body is desirable to ascertain. Non-limiting examples of such analytes include d-dimer, fibrin monomer, troponin I, troponin T, fibrinpeptide A, creatine kinase MB (CK-MB), fatty acid binding protein, carbonic anhydrase III, F 1.2, tissue factor, tissue factor pathway inhibitor, thrombin anti-thrombin complex, tissue plasminogen activator (t-PA), plasminogen activator inhibitor-l (PAI-1), t-PA-PAI-1 complex, plasmin antiplasmin complex, beta chain peptides 15-42, platelet factor 4, beta thromboglobulin, endothelin, bradykinin, anti-phospholipid antibody, Von Willebrand factor, myoglobin, brain natriuretic peptide, hepatitis B antigen, hepatitis C antigen (core), botulinum toxin, interleukin 6 (IL-6) and erythropoetin.

As used herein, reagents containing an “autologous analyte” refers to reagents for use in assays which contain an analyte which is identical, substantially similar to, or a recombinant form of the target analyte for which the sample is being assayed. An autologous analyte is considered to be “substantially similar to” the target analyte if it contains portions of the target analyte (e.g., epitopes) which, after addition to the reagent, produce a reagent which lowers the coefficient of variation of a non-competitive assay by at least about 10%, preferably at least about 20%, and most preferably, at least about 50% relative to the non-competitive assay run without a reagent containing an autologous analyte.

As used herein, an “immunoassay” is an assay that utilizes an antibody or antigen to specifically bind to the analyte. The immunoassay is characterized by the use of specific binding to a particular antibody or antigen to detect, isolate, target, and/or quantify the analyte in a sample.

The term “optimization” refers to the process of designing or developing an assay system so as to improve one or more performance characteristics of the assay, such as improving its precision within a particular working range of analyte concentrations, i.e. minimizing the random error incurred in the measurement of analyte concentration. This process may involve improving the sensitivity of the assay, that is the ability of the assay system to determine low concentrations of analytes, represented numerically by the lowest analyte concentration distinguishable from zero (the “detection limit”).

### DETAILED DESCRIPTION OF THE INVENTION

The present invention provides assays and kits for detecting or determining the concentration of an analyte in a biological sample. The assay, kits and reagents of the present invention contain an amount of a target analyte derived from a source other than the sample which reduces the coefficient of variation of a non-competitive assay and thereby increases the precision and confidence level of the assay. Thus, the assays and kits of the present invention have improved precision by which it is possible to measure low dosages of an analyte in a biological sample.

The present invention is useful in assaying for a wide variety of analytes in virtually any type of sample which is liquid, which can be liquified, which can be suspended in a 2.5 liquid or which can be dissolved in a liquid. The assay and kit will find their greatest use with biological specimens, such as blood, serum, plasma, urine, cerebral fluid, spinal fluid, ocular lens liquid (tears), saliva, sputum, semen, cervical mucus, scrapings, swab samples, and the like. Under certain circumstances, it may be desirable to pretreat the biological sample, such as by separation, dilution, concentration, filtration, chemical treatment, or a combination thereof, prior to assaying the sample.

The present invention is directed to assays with increased sensitivity and precision thereby allowing the detection of low dosages of an analyte in a sample without adversely affecting the long linear range of the immunoassay. Therefore, one aspect of the invention provides processes for determining low dosages of the target analyte while reducing the coefficient of variation of a non-competitive assay by at least about 10%, preferably at least about 20%, and most preferably, at least about 50% relative to non-competitive assays known in the art. Preferably, the immunoassay can be used to determine the concentration of troponin I in the range of about 0.1 to about 0.8 ng/ml in a sample without affecting the long linear range of the assay. In another embodiment, the immunoassay can determine the concentration of d-dimer in the range of about 70 ng/ml to about 6000 ng/ml without affecting the long linear range of the assay. Preferably, in the immunoassays of the present invention, the signal for low dosages of the target analyte is increased by at least about 50% of the expected value. Low dosages of target analyte are preferably about 70 to about 150 ng/ml of d-dimer or about 0.2 to about 0.8 ng/ml of troponin I. The expected value is the value from a dosage-response curve generated by an assay run using a reagent without autologous analyte wherein the value is greater than zero.

In another aspect of the invention, the assay can be used to differentiate between about 12 ng/ml and 20 ng/ml of d-dimer in a sample. However, in differentiating between such low levels of analyte, the autologous analyte (in this case, d-dimer) would need to be added in an amount which may decrease the overall utility of the assay by lowering the highest point of the reportable range for the calibration curve.

However, in some cases, the presence of any amount of target analyte in a patient sample may be significant in determining management options for a patient e.g., the presence of certain cardiac markers may be indicative of a cardiac event (e.g., troponin I, troponin T), infectious diseases (e.g., hepatitis C, Hepatitis B), or toxin (e.g., botulinum toxin, diptheria, toxin). In these cases, the level of sensitivity for an assay may be adjusted depending on the area of the assay which would provide the most useful information.

A variety of non-competitive immunoassays can be used for the processes of the present invention to detect or determine the concentration of a target analyte in a biological sample. Well-characterized assays such as agglutination assays; enzyme immunoassays (see calorimetric assays below); two-site immunometric assay such as direct immunoassays, radio immunoassays, fluorescent immunoassays (fluorescent tags on detection antibodies, or on secondary antibodies, e.g., anti mouse antibodies), chemiluminescent assays and calorimetric assays using horse radish peroxidase, alkaline phosphatase, urease, beta galactosidase and suitable substrates; binding assays such as plasmon resonance; energy transfer assays; lateral flow assays; and flow cytometry assays are all contemplated in this invention.

The two-site immunometric assay method is commonly referred to as a “sandwich immunoassay” method. In immunometric assays, two anti-analyte antibodies are employed. One of the anti-analyte antibodies is labeled (the “detection antibody”) and the other is immobilized or immobilizable (the “capture antibody”). As is known in the art, the capture and detection antibodies can be contacted simultaneously or sequentially with the test sample. Sequential methods can be accomplished by incubating the capture antibody with the sample, and adding the labeled antibody a predetermined time thereafter (sometimes referred to as the “forward” method); or the detection antibody can be incubated with the sample first and then the labeled antibody added (sometimes referred to as the “reverse” method). After the necessary incubation(s) have occurred, to complete the assay, the capture antibody is separated from the liquid test mixture, and the label is measured in at least a portion of at least one of the separated capture antibody phase or the remainder of the liquid test mixture, normally the former since it comprises the target bound by or “sandwiched” between the capture and detection antibodies.

Typically, one or both of the capture and detection antibodies are monoclonal antibodies. The label used in the detection antibody can be selected from any of those known conventionally in the art. Commonly, the label is an enzyme or a chemiluminescent moiety (as in a chemiluminescent assay), but can also be a radioactive isotope (as in a radio immunoassay), a fluorophor (as in a fluorescent immunoassay), a detectable ligand e.g., detectable by a secondary binding by a labeled binding partner for the ligand, and the like. A desirable property of the capture antibody is that it provides a means for being separated from the remainder of the test mixture. Accordingly, as is understood in the art, the capture antibody can be introduced to the assay in an already immobilized or insoluble form, or can be in a immobilizable form, i.e., a form which enables immobilization or separation to be accomplished subsequent to introduction of the capture antibody to the assay. Examples of immobilized capture antibody are antibody covalently or noncovalently attached to a solid phase such as a magnetic particle, a latex particle, a microtiter plate well, a bead, a cuvette, a pipette or other reaction vessel. An example of an immobilizable capture antibody is antibody which has been chemically modified with a ligand moiety, e.g., a hapten, biotin., or the like, and which can thus be subsequently immobilized by contact with an immobilized (as described above for directly immobilized capture antibody) form of a binding partner for the ligand, e.g., an antibody, avidin, or the like.

In an energy transfer assay, the measured interaction is usually a change or shift in light emission, which is caused by the transfer of light, or energy, from one label to a second proximately located label. The label from which the energy is transferred is referred to as the “donor label”, while the label to which the energy is transferred is referred to as the “acceptor label”.

In one embodiment, the assay is performed using microtiter well plates. In this method, the wells of microtiter plates are coated with the antibody mixture described herein and the wells are dried or preserved. Biological samples to be tested are then prepared at various dilutions in the reagent containing the autologous analyte and added to the wells. After a period of incubation, the biological sample is removed and the wells are washed to remove any unbound analyte. Then a preparation containing labeled antibody which binds to the analyte is added and the wells are further incubated. The labeled antibody preparation is again discarded and another wash is performed to remove any unbound labeled antibody. If the target analyte is present in the biological sample, then analyte bind to the antibodies bound to the well to form antibody-analyte complexes. The labeled antibody will react with the antibody-analyte and produce a color change that reflects the quantity of analyte present.

In another embodiment, the immunoassay is performed using an agglutination assay and more preferably, a latex agglutination assay. Generally, in a latex agglutination assay, the antibodies are affixed to latex beads to form antibody-coated latex beads. The biological sample is then incubated directly with the latex particles. In a short time, the reaction is examined for the presence of cross-linked, or agglutinated latex particles which indicate the presence of the target analyte.

In the assay of the present invention, a sample suspected of containing the analyte is combined with a reagent comprising the target analyte, preferably a purified analyte, which is derived from a source other than the sample being assayed to form an assay mixture. Thus, the reagent of the present invention contains an amount of autologous analyte which is derived from a source other than the sample being assayed. Further, the autologous analyte is dissolved in a solution other than the sample prior to combining the reagent with the sample.

Preferably, the reagent contains an amount of autologous analyte which lowers the coefficient of variation of a non-competitive assay by at least about 10%, preferably at least about 20%, and most preferably, at least about 50%.

In one embodiment, autologous analyte is added to the reagent in an amount to increase the signal of the immunoassay for low dosages of the target analyte by at least about 50% of the expected value. Low dosages of the target analyte which may be detected using the immunoassay are preferably about 70 to about 150 ng/ml of d-dimer and about 0.2 to about 0.8 ng/ml of troponin I. The expected value is the value from a calibration curve generated from an assay run using a reaction buffer without the addition of autologous analyte wherein the value is greater than zero.

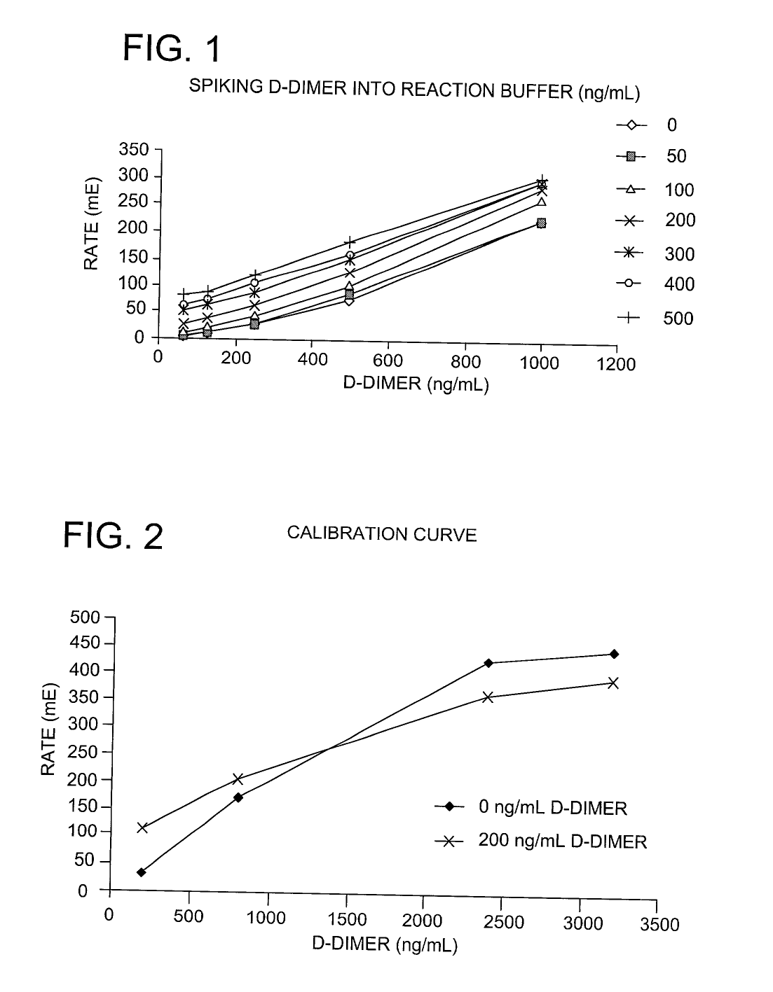

As indicated earlier, the sample may include low levels of a protein or other moiety unrelated to the target analyte which participates in non-specific binding reactions with the various components of the assay (e.g., the antibody). Without being limited to any particular theory, the addition of the autologous analyte to the reagent acts as a priming system by providing enough autologous analyte to raise the signal above the background noise caused by such non-specific binding. Assays run using a reagent without the addition of the autologous analyte result in a calibration curve which provides little or low signal for low dosages of the analyte in the sample (see FIGS. 1 and 2) and results in poor sensitivity and precision of the assay at low target analyte concentrations. However, addition of the autologous analyte to the reagent improves the specific target analyte-antibody associated signal by lowering the threshold of proteins or other moieties which may participate in non-specific binding reactions. In doing so, the immunological reaction between the antibody-coated particles and analyte required for crosslinking can occur, thereby resulting in the formation of a microparticle complex of sufficient size to initiate light scattering.

Thus, adding an amount of autologous analyte to the reagent provides a detectable signal for low dosages of the target analyte and in essence, amplifies the signal obtained for low dosages of target analyte in the sample. The signal can then be related through a normal calibration curve to the concentration of target analyte within the sample. Preferably, the presence of the analyte will not be detectable since the amount of autologous analyte added is preferably made consistent through the consistent dilution of the patient sample with the reaction buffer containing the autologous analyte.

Further, the signal obtained for low concentrations of target analyte may be low because a certain amount of target analyte is required to occupy binding sites on the antibody-coated microparticles of the agglutination assay before a signal is registered by assay instrumentation. However, the autologous analyte is not added to the reagent in a amount to saturate the binding sites on the antibody-coated particles since this would result in a reduction of signal due to the antigen excess. Saturation of the antibody binding sites would result in the inability of the particles to agglutinate through antigen bridging.

Preferably, the reagent is a reaction buffer used to dilute the sample prior to incubation with the antibody. Typically, agglutination assays employ two buffers. A storage buffer is used for the particles which tends to be of a lower molarity in order to keeps particles apart, i.e., colloidally stable. The reaction buffer is usually a different pH and has a higher molarity, typically to overcome the stability of the storage buffer so that the immunological reaction between the analytes and antibodies can occur. Detergents are added to the buffers to provide charge and solubility characteristics and to prevent non-specific binding. Microtiter plate assays typically use the same buffer systems. Examples of buffer systems utilized in assays include borate, barbital (veronal), MES, Tris, bis, bicine, hepes, taps, sodium phosphate, potassium phosphate, imidazole, or any buffer system with a pKa of between 5.5 and 8.5.

Preferably, the reaction buffer used is a MES or bis buffer. Preferably, the amount of autologous analyte added to the reaction buffer will depend on the target analyte being assayed for in the sample. However, the amount of autologous analyte added to the reaction buffer is sufficient to maintain the linearity of the curve and results in the amplification of the signal at low concentrations of the target analyte. However, the addition of autologous analyte to the reagent, preferably a reaction buffer, is not in an amount which upon incubating with the antibody mixture described herein, would result in the saturation of the binding sites on the antibody-coated particles. Saturation of the binding sites on the antibodies would result in a reduction of signal due to the antigen excess due to the inability of the particles to agglutinate through antigen bridging.

The particular target analyte of the assay is not critical to the invention and it will be appreciated that the immunoassay may be used to assay a sample for a number of various analytes. Hence, the reagent may contain an autologous analytes such as d-dimer, fibrin monomer, troponin I, troponin T, fibrinpeptide A, creatine kinase MB (CK-MB), fatty acid binding protein, carbonic anhydrase III, F 1.2, tissue factor, tissue factor pathway inhibitor, thrombin anti-thrombin complex, tissue plasminogen activator (t-PA), plasminogen activator inhibitor-1 (PAI-1), t-PA-PAI-1 complex, plasmin antiplasmin complex, beta chain peptides 15-42, platelet factor 4, beta thromboglobulin, endothelin, bradykinin, anti-phospholipid antibody, Von Willebrand factor, myoglobin, brain natriuretic peptide, hepatitis B antigen, hepatitis C antigen (core), botulinum toxin, interleukin 6 (IL-6) and erythropoetin. Preferred autologous analytes for use in the reagent are d-dimer, fibrin monomer, fibrinpeptide A, troponin I, troponin T, CK-MB, carbonic anhydrase III or fatty acid binding protein.

The amount of autologous analyte added to the reagent, preferably a reaction buffer and more preferably, a MES or bis buffer, will depend on the target analyte being assayed for in the sample. In a preferred embodiment, fibrin derivatives, more preferably, purified or recombinant d-dimer and fragment D, are used in the reagent. The amount of the fibrin derivative in the reaction reagent is about 100 to about 1000 ng/ml, preferably, about 200 to about 600 ng/ml, more preferably, about 200 to about 400 ng/ml, and even more preferably, about 200 ng/ml. In another preferred embodiment, troponin I, preferably a purified troponin I or recombinant troponin I, is the autologous analyte used to produce the reagent. The amount of the purified troponin I added to the reaction reagent is about 0.5 to about 10 ng/ml, preferably, about 1 to about 6 ng/ml, and more preferably, about 2.5 to about 4 ng/ml and even more preferably, about 3 ng/ml.

This assay mixture is incubated with an antibody mixture to allow binding reactions between the analyte and antibody to occur. The antibody mixture contains antibodies which bind to the target analyte. The antibodies in the antibody mixture may be polyclonal antibodies or monoclonal antibodies but preferably, monoclonal antibodies are utilized. In one embodiment, the antibody mixture contains antibodies specific for at least two epitopes on the target analyte.

Preferably, a surface is provided to which the antibody is bound such as microparticles such as latex particles; microtiter plate wells which also serve as the container for the assay mixture; or magnetic particles which can be separated in a magnetic field gradient. Various types of microparticles allow for the customization of particle coating to accommodate specific assay needs, such as sensitivity. Examples of different microparticles for use in agglutination assays include polystyrene particles, streptavidin coated particles, anti-mouse coated particles, protein A coated particles, dye incorporated particles, carboxylate-modified particles and various chemical modified particles e.g., preactivated beads for coupling to protein (activated with amino, hydroxyl, hydrazide, amide, chloromethyl, epoxy, and aldehyde).

In one embodiment of the present invention, the antibody is preferably immobilized on a microparticle bead, preferably a latex bead, thereby forming an antibody-coated bead. Preferably, the microparticle beads will be latex beads such as polystyrene, polyacrylate, polyacetate, polyvinylchlorite and polyurethane teflon. Generally, the size of microparticles i.e., the diameter of the particle should be about one third of the wavelength used in the analyzer. For example, if an analyzer reads at a wavelength of 600 nm is used, the latex bead size should be approximately 200 nm. For analyzers which read from 300-800 nm, bead sizes from 50 to 300 nm may be used. In one embodiment, the latex beads are preferably 50 to 300 nm, preferably 70 to 150 nm, and most preferably, 120 nm in size. The beads will usually be approximately uniform in size and will have either a rough or smooth surface, preferably smooth. Preferably the beads are rounded or oblong, preferably, round and have surface properties which minimize non-specific binding. The amount of antibody coupled to the bead will depend on several factors such as the size of the bead, the parking area of the bead and the target analyte. Those of skill in the art will be able to design or modify antibody coated microparticles suitable for used in an immunoassay for a particular target analyte.

After allowing the assay mixture and the antibody mixture to incubate, the immunological reaction between the antibodies and target analyte result in the formation of large aggregates. These large aggregates result in a change in the light scatter of the solution and are capable of measurement by nephelometric or turbidimetric methods. In turbidimetry, the reduction of light transmitted through the suspension of particles, or aggregates, is measured. The reduction is caused by reflection, scatter, and absorption of the light by the aggregates. In nephelometry, it is the light scattered or reflected toward a detector that is not in the direct path of light which is measured. In both turbidimetry or nephelometry, the rate of change in light scatter may also be measured as an indication of the amount of antigen present.

In a preferred embodiment, turbidimetric measurement is utilized for the immunological agglutination reactions and is preferred since no special equipment is required other than a spectrophotometer. The spectrophotometer measures increased absorbance which is due to the increasing particle size resulting from the agglutination reaction. This increased absorbance is a direct measure of the agglutination caused by the analyte. When light is passed through an agglutinated reaction mixture, part of the incident radiant energy is dissipated by absorption, reflection, and refraction, while the remainder is transmitted. Measurement of the intensity of the transmitted light as a function of the concentration of the dispersed phase is the basis of turbidimetric analysis.

This invention also contemplates kits for practice of the above-described processes for detecting or determining the concentration of a target analyte. Accordingly, the reagent, antibody mixture and other assay components necessary are provided in the form of a test kit, that is, in a packaged collection or combination as appropriate for the needs of the user and any analytical instrumentation involved. Minimally, the kit will comprise the reagent containing the autologous analyte and an antibody mixture as described above. The kit can of course include appropriate packaging, containers, labeling, buffers, controls, calibrators and indicators for detecting or determining the concentration of a target analyte in a sample.

The following examples illustrate the principles and advantages of the invention.

### EXAMPLES

### Example 1

### Preparation of D-Dimer for Addition to Reaction Buffer

Thirty milliliters of pooled normal human plasma were treated with 0.5 ml of Sigma thrombin reagent 886-12, and incubated at 37 C. overnight. Clotting occurred within 30 minutes. Alternatively, addition of 0.5-2.0 ml of APTT reagent and 3 ml of 0.02M calcium chloride solution to the plasma would provide a clot after overnight incubation. The clot was separated from the serum, washed three times in saline solution, and dried on a paper towel. 10 ml of saline was added for every 1.5g wet weight of clot, and one unit of Sigma plasmin (Sigma catalog #P-4895) was added per 10 ml of solution. The solution was incubated at 37° C. for 72 hours. The resulting solution was treated with aprotinin (100U) to neutralize any remaining plasmin activity, and filtered through a 0.2 micron filter. The d-dimer material is stable in this form at 2-8° C. for 24 months.

The D-dimer solution was diluted 1/1000 in saline and tested for d-dimer content using a quantitative d-dimer assay (Assocachrome, IL kit). The results were multiplied by 1000. A typical yield for this procedure would be about 5-7 ml of liquid with a d-dimer content of 1,300,000 ng/ml.

### Example 2

### Preparation of Reaction Buffer and Effect on Low Concentrations of D-Dimer in Sample

The reaction buffer was prepared by adding MES for a final concentration of 0.45M MES, pH 7.0. Human D-dimer was prepared as described in Example 1 and added at varying concentrations ranging from 0 to 500 ng/ml.

The reaction buffer was used with microparticles coated with monoclonal antibody MA 8D3. An assayed D-dimer calibrator was diluted to give D-dimer concentrations ranging from 62.5 to 1000 ng/mL. These dilutions were analyzed on the AMAX 190 Plus analyzer. The analyzer is programmed to automatically dispense 17uL of sample into a cuvette. 58uL of reaction buffer was added to the sample and the mixture was incubated for 60 seconds. The mixture was transferred to the photo-optical path and 75uL of antibody-coated latex beads was dispensed into the cuvette. A Reading was initiated after 12 seconds and completed after 5 minutes.

The results and data of this study are depicted in FIG. 1. The difference in absorbance is reported as mE units.

### Example 3

### Effect of D-Dimer-Spiked Reaction Buffer on Full Calibration Curve

The reaction buffer was prepared by adding MES for a finial concentration of 0.45M MES. Bovine Serum Albumin (BSA) was added as a stabilizer at final concentration of 2.0% weight/volume. The final pH was 7.0. Human D-dimer was prepared as described in Example 1 and a final concentration of 200 ng/mL was added to a portion of the buffer.

An assayed d-dimer calibrator was reconstituted and diluted to varying concentrations of D-dimer ranging from 200 to 3200 ng/mL. These dilutions were analyzed on the AMAX 190 Plus analyzer. 30uL of sample was dispensed into a cuvette. 50uL of reaction buffer, with or without added d-dimer was added to the sample and the mixture was incubated for 20 seconds. The mixture was transferred to the photo-optical path and 70uL of latex coated with 8D3 antibody was dispensed into the cuvette. Reading was initiated after 5 seconds and completed after 5 minutes.

The results and data of this study are depicted in FIG. 2. The difference in absorbance is reported as mE units.

### Example 4

### Effect of D-Dimer-Spiked Reaction Buffer on Assay Precision at Cut-Off Levels

Using the reagents and protocol from Example 3, precision studies were performed on the diluted calibrator representing a concentration close to the critical cutoff level. A precision study performed on a sample representing a critical value for the d-dimer assay. Sample values were assigned using different calibration curves. As can be seen in Table 1, the % CV for the assays with the added d-dimer were less than 50% of those for samples without the added d-dimer.

TABLE 1

| Reaction Buffer | |
|---|---|
| 0 ng/ml D-dimer | 200 ng/mL D-dimer |

| Run | mE | ng/mL | mE | ng/mL |
|---|---|---|---|---|
| 1 | 27.6 | 171 | 113.5 | 193 |
| 2 | 27.5 | 170 | 110.4 | 179 |
| 3 | 26.4 | 164 | 116.1 | 205 |
| 4 | 26.6 | 165 | 116.2 | 205 |
| 5 | 31.6 | 191 | 115.7 | 203 |
| 6 | 20.1 | 131 | 114.7 | 198 |
| 7 | 29.3 | 180 | 116.7 | 208 |
| 8 | 27.3 | 169 | 112.4 | 188 |
| 9 | 26.3 | 164 | 115.1 | 200 |
| 10 | 27.6 | 171 | 115.5 | 202 |
| Mean | 27.03 | 167.60 | 114.63 | 198.10 |
| SD | 2.91 | 15.29 | 1.98 | 9.00 |
| % CV | 10.76 | 9.12 | 1.72 | 4.54 |

### Example 5

### Effect of D-Dimer-Spiked Reaction Buffer on Assay Precision at Low Level Analyte Concentration

Using the reagents and protocol from Example 3, precision studies were performed on the calibrator diluent for IL Test D-Dimer (prod # 20008500) a buffer containing phosphate buffer (as opposed to MES as used in previous examples), bovine serum albumin, stabilizers and preservatives. The diluent was used with no added d-dimer or with 200ng/ml of d-dimer.

Table 2 is a precision study performed on a sample representing a zero level of d-dimer and 200 ng/ml d-dimer. Sample values are given in delta mE, from the Amax 190. As can be seen in Table 2, the % CV obtained for the assays run with the reaction buffer with the added d-dimer was lowered by more than 50% relative to those for samples diluted in reaction buffer in the absence of added d-dimer.

TABLE 2

| Test | 0 ng/ml D-dimer | 200 ng D-Dimer |
|---|---|---|
| 1.00 | 5.60 | 24.50 |
| 2.00 | 1.40 | 21.20 |
| 3.00 | 4.00 | 33.30 |
| 4.00 | 1.20 | 22.70 |
| 5.00 | 2.80 | 33.90 |
| 6.00 | 0.70 | 27.90 |
| 7.00 | 6.50 | 33.30 |
| 8.00 | 2.50 | 21.90 |
| 9.00 | 6.20 | 21.20 |
| 10.00 | 0.60 | 22.80 |
| 11.00 | 0.10 | 26.00 |
| 12.00 | 6.10 | 25.30 |
| 13.00 | 0.70 | 27.50 |
| 14.00 | 1.00 | 25.90 |
| 15.00 | 1.10 | 26.10 |
| 16.00 | 1.80 | 24.60 |
| Sum | 42.30 | 418.10 |
| Mean | 2.64 | 26.13 |
| SD | 2.28 | 4.19 |
| % CV | 86.10 | 16.03 |

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Further, the specific embodiments of the present invention as set forth are not intended as being exhaustive or limiting of the invention, and that many alternatives, modifications, and variations will be apparent to those skilled in the art in light of the foregoing examples and detailed description. Accordingly, this invention is intended to embrace all such alternatives, modifications, and variations that fall within the spirit and scope of the following claims. While some of the examples and descriptions above include some conclusions about the way the invention may function, the inventors do not intend to be bound by those conclusions, but puts them forth only as possible explanations.

We claim:

1. A process for detecting or determining the concentration of a target analyte in a sample, said process comprising the steps of:
a. combining the sample with a reagent to form an assay mixture, the reagent comprising target analyte from a source other than the sample;
b. incubating the assay mixture with an antibody mixture comprising antibodies which bind at least two non-overlapping epitopes on the target analyte to form analyte-antibody complexes; and
c. examining the incubated assay mixture for analyte-antibody complexes.

2. The process of claim 1, wherein the reagent comprises target analyte in an amount which lowers a percent coefficient of variation for the target analyte in a non-competitive assay by at least about 10% relative to the non-competitive immunoassay run without the target analyte derived from a source other than the sample.

3. The process of claim 2, wherein the reagent comprises target analyte in an amount which lowers a percent coefficient of variation for the target analyte in a non-competitive assay by at least about 20%.

4. The process of claim 3, wherein the reagent comprises target analyte in an amount which lowers a percent coefficient of variation for the target analyte in a non-competitive assay by at least about 50%.

5. The process of claim 1, wherein the target analyte derived from a source other than the sample is d-dimer, fibrin monomer, fibrinpeptide A, troponin I, troponin T, CK-MB, carbonic anhydrase III or fatty acid binding protein.

6. The process of claim 5 wherein the target analyte derived from a source other than the sample comprises at least one fibrin derivative.

7. The process of claim 6 wherein the at least one fibrin derivative is fibrin d-dimer and fragment D from fibrin.

8. The process of claim 7 wherein the reagent comprises about 100 to 1000 ng/ml of the at least one fibrin derivative.

9. The process of claim 8 wherein the reagent comprises about 200 to 600 ng/ml of the at least one fibrin derivative.

10. The process of claim 9 wherein the reagent comprises about 200 to 400 ng/ml of the at least one fibrin derivative.

11. The process of claim 10 wherein the reagent comprises about 200 ng/ml of the at least one fibrin derivative.

12. The process of claim 5 wherein the target analyte derived from a source other than the sample is troponin I.

13. The process of claim 12 wherein the reagent comprises about 0.5 to about 10 ng/ml of troponin I.

14. The process of claim 13 wherein the reagent comprises about 1 to about 6 ng/ml of troponin I.

15. The process of claim 14 wherein the reagent comprises about 2.5 to about 4 ng/ml of troponin I.

16. The process of claim 15 wherein the reagent comprises about 3 ng/ml of troponin I.

17. The process of claim 1, wherein the antibody mixture comprises monoclonal antibodies or fragments thereof.

18. The process of claim 17, wherein the monoclonal antibodies bind to cross-linked fibrin derivatives and fragment D from non-crosslinked fibrin.

19. The process of claim 18, wherein the monoclonal antibodies bind to d-dimer and fragment D from non-crosslinked fibrin.

20. The process of claim 17, wherein the antibody mixture is immobilized on latex particles or microtiter well plates.

21. The process of claim 20, wherein the latex particles are 120 nm latex beads.

22. A kit for conducting a non-competitive immunoassay for detecting or determining the concentration of a target analyte in a sample, the kit comprising:
a. a reagent comprising the target analyte derived from a source other than the sample; and
b. an antibody mixture comprising antibodies which bind at least two non-overlapping epitopes on the target analyte.

23. The kit of claim 22 wherein the reagent comprises the target analyte from a source other than the sample in an amount which lowers a percent coefficient of variation for the target analyte in the non-competitive assay by at least about 10% relative to the non-competitive immunoassay run without analyte derived from a source other than the sample.

24. The kit of claim 23, wherein the reagent comprises target analyte in an amount which lowers a percent coefficient of variation for the target analyte in a non-competitive assay by at least about 20%.

25. The kit of claim 24, wherein the reagent comprises target analyte in an amount which lowers a percent coefficient of variation for the target analyte in a non-competitive assay by at least about 50%.

26. The kit of claim 22 wherein the target analyte derived from a source other than the sample is d-dimer, fibrin monomer, fibrinpeptide A, troponin I, troponin T, CK-MB, carbonic anhydrase III or fatty acid binding protein.

27. The kit of claim 26 wherein the target analyte derived from a source other than the sample comprises at least one fibrin derivative.

28. The kit of claim 27 wherein the at least one fibrin derivative is fibrin d-dimer and fragment D from fibrin.

29. The kit of claim 28 wherein the reagent comprises about 100 to 1000 ng/ml of the at least one fibrin derivative.

30. The kit of claim 29 wherein the reagent comprises about 200 to 600 ng/ml of the at least one fibrin derivative.

31. The kit of claim 30 wherein the reagent comprises about 200 to 400 ng/ml of the at least one fibrin derivative.

32. The kit of claim 31 wherein the reagent comprises about 200 ng/ml of the at least one fibrin derivative.

33. The kit of claim 26 wherein the target analyte derived from a source other than the sample is troponin I.

34. The kit of claim 33 wherein the reagent comprises about 0.5 to about 10 ng/ml of troponin I.

35. The kit of claim 34 wherein the reagent comprises about 1 to about 6 ng/ml of troponin I.

36. The kit of claim 35 wherein the reagent comprises about 2.5 to about 4 ng/ml of troponin I.

37. The kit of claim 36 wherein the reagent comprises about 3 ng/ml of troponin I.

38. The kit of claim 22, wherein the antibody mixture comprises monoclonal antibodies or fragments thereof.

39. The kit of claim 38, wherein the monoclonal antibodies bind to cross-linked fibrin derivatives and fragment D from non-crosslinked fibrin.

40. The kit of claim 39, wherein the monoclonal antibodies bind to d-dimer and fragment D from non-crosslinked fibrin.

41. The kit of claim 22, wherein the antibody mixture comprises antibodies which bind to troponin I.

42. The kit of claim 22, wherein the antibody mixture is immobilized on latex particles or microtiter well plates.

43. The kit of claim 42, wherein the latex particles are 120 nm latex beads.

44. A kit for conducting a non-competitive immunoassay for detecting or determining the concentration of a target analyte in a sample, the kit comprising:
a. a reagent comprising the target analyte derived from a source other than the sample; and
b. an antibody mixture comprising antibodies immobilized on latex particles, wherein the antibodies bind to the target analyte.

45. The kit of claim 44 wherein the reagent comprises the target analyte from a source other than the sample in an amount which lowers a percent coefficient of variation for the target analyte in the non-competitive assay by at least about 10% relative to the non-competitive immunoassay run without analyte derived from a source other than the sample.

46. The kit of claim 45, wherein the reagent comprises target analyte in an amount which lowers a percent coefficient of variation for the target analyte in a non-competitive assay by at least about 20%.

47. The kit of claim 46, wherein the reagent comprises target analyte in an amount which lowers a percent coefficient of variation for the target analyte in a non-competitive assay by at least about 50%.

48. The kit of claim 44 wherein the target analyte derived from a source other than the sample is d-dimer, fibrin monomer, fibrinpeptide A, troponin I, troponin T, CK-MB, carbonic anhydrase III or fatty acid binding protein.

49. The kit of claim 48 wherein the target analyte derived from a source other than the sample comprises at least one fibrin derivative.

50. The kit of claim 49 wherein the at least one fibrin derivative is fibrin d-dimer and fragment D from fibrin.

51. The kit of claim 50 wherein the reagent comprises about 100 to 1000 ng/ml of the at least one fibrin derivative.

52. The kit of claim 51 wherein the reagent comprises about 200 to 600 ng/ml of the at least one fibrin derivative.

53. The kit of claim 52 wherein the reagent comprises about 200 to 400 ng/ml of the at least one fibrin derivative.

54. The kit of claim 53 wherein the reagent comprises about 200 ng/ml of the at least one fibrin derivative.

55. The kit of claim 45 wherein the target analyte derived from a source other than the sample is troponin I.

56. The kit of claim 55 wherein the reagent comprises about 0.5 to about 10 ng/ml of troponin I.

57. The kit of claim 56 wherein the reagent comprises about 1 to about 6 ng/ml of troponin I.

58. The kit of claim 57 wherein the reagent comprises about 2.5 to about 4 ng/ml of troponin I.

59. The kit of claim 58 wherein the reagent comprises about 3 ng/ml of troponin I.

60. The kit of claim 45, wherein the antibody mixture comprises monoclonal antibodies or fragments thereof.

61. The kit of claim 60, wherein the monoclonal antibodies bind to cross-linked fibrin derivatives and fragment D from non-crosslinked fibrin.

62. The kit of claim 61, wherein the monoclonal antibodies bind to d-dimer and fragment D from non-crosslinked fibrin.

63. The kit of claim 45, wherein the antibody mixture comprises antibodies which bind to troponin I.

64. The kit of claim 60, wherein the antibody mixture is immobilized on latex particles or microtiter well plates.

65. The kit of claim 64, wherein the latex particles are 120 nm latex beads.

66. A reagent for a non-competitive immunoassay for an analyte in a sample, the reagent comprising the analyte derived from a source other than the sample dissolved in a solution other than the sample, the analyte being dissolved in the solution in an amount which lowers a percent coefficient of variation for the analyte in the non-competitive assay by at least about 10% relative to the non-competitive immunoassay run without analyte derived from a source other than the sample.

67. The reagent of claim 66, wherein the amount of the analyte lowers the percent coefficient of variation of the analyte in the non-competitive assay by at least about 20%.

68. The reagent of claim 67, wherein the amount of the analyte lowers the percent coefficient of variation of the analyte in the non-competitive assay by at least about 50%.

69. The reagent of claim 66 wherein the analyte derived from a source other than the sample is d-dimer, fibrin monomer, fibrinpeptide A, troponin I, troponin T, CK-MB, carbonic anhydrase III or fatty acid binding protein.

70. The reagent of claim 69 wherein the analyte derived from a source other than the sample is at least one fibrin derivative.

71. The reagent of claim 70 wherein the at least one fibrin derivative is fibrin d-dimer and fragment D of fibrin.

72. The reagent of claim 71 wherein the reagent comprises about 100 to 1000 ng/ml of the at least one fibrin derivative.

73. The reagent of claim 72 wherein the reagent comprises about 200 to 600 ng/ml of the at least one fibrin derivative.

74. The reagent of claim 73 wherein the reagent comprises about 200 to 400 ng/ml of the at least one fibrin derivative.

75. The reagent of claim 74 wherein the reagent comprises about 200 ng/ml of the at least one fibrin derivative.

76. The reagent of claim 69 wherein the analyte derived from a source other than the sample is troponin I.

77. The reagent of claim 76 wherein the reagent comprises about 0.5 to about 10 ng/ml of troponin I.

78. The reagent of claim 77 wherein the reagent comprises about 1 to about 6 ng/ml of troponin I.

79. The reagent of claim 78 wherein the reagent comprises about 2.5 to about 4 ng/ml of troponin I.

80. The reagent of claim 79 wherein the reagent comprises about 3 ng/ml of troponin I.
